(19) United States
(12) Patent Application Publication
(10) Pub. No.: US 2017/0215750 A1
(43) Pub. Date: Aug. 3, 2017

(54) LOW VOLUME BLOOD PRESSURE METER AND CUFF THEREOF

(71) Applicant: NEC Corporation, Tokyo (JP)

(72) Inventors: Ersin ALTINTAS, Tokyo (JP); Masahiro KUBO, Tokyo (JP); Katsumi ABE, Tokyo (JP); Kimiyasu TAKOH, Tokyo (JP); Yuji OHNO, Tokyo (JP); Osamu TOCHIKUBO, Kanagawa (JP)

(73) Assignee: NEC Corporation, Tokyo (JP)

(21) Appl. No.: 15/500,484

(22) PCT Filed: Aug. 7, 2014
(86) PCT No.: PCT/JP2014/071416

§ 371 Date: Jan. 30, 2017

(51) Int. Cl. A61B 5/022; A61B 5/021; A61B 5/02; A61B 5/0235

(52) U.S. Cl. CPC A61B 5/02241; A61B 5/02225; A61B 5/0235; A61B 5/02233; A61B 5/022; A61B 5/021; A61B 5/02

(57) ABSTRACT

A low volume or narrower blood pressure cuff compared to commercial counterparts with similar medical approval accuracy is introduced. The present invention comprises an occlusion component configured to occlude the artery, and a pulse wave detection component to detect blood pressure oscillations. Blood pressure readings within medical approval accuracy are achieved by controlling and adjusting fluid amount in or fluid flowing into said pulse wave detection component.

### TECHNICAL FIELD

This invention is related to a blood pressure meter and cuff.

### BACKGROUND ART

Blood pressure is one of the vital signs (i.e. blood pressure, breathe, temperature, heart pulse etc.) in humans or animals, and it is one of the strongest parameters to monitor and to diagnose the medical conditions and the diseases such as heart diseases and hypertension. For a reliable medical evaluation and treatment, blood pressure measurement accuracy less than ±5 mmHg is necessary from a body portion. Since, the blood pressure value is strongly dependent on the vertical distance from the heart level; blood pressure measurement from upper arm at the level of heart is universally recognized by medical professionals for a more reliable and accurate measurement. This is generally achieved by a structure called “cuff”, which is wrapped (or placed) around upper arm of human.

Usual cuffs are composed of bags or bladders inflated/deflated (or pressurized/depressurized) by air through a pressure control unit. In order to measure the blood pressure, there can be different methods such as (i) detection of Korotkoff sounds usually achieved by a stethoscope by medical professionals, (ii) oscillometric techniques detecting the oscillations in the inflatable air bag due to pressure oscillations caused by artery, and (iii) techniques depending on Doppler Effect. Korotkoff sounds and oscillometric detections are widely accepted and employed in commercial blood pressure monitors, meters or devices (i.e. sphygmomanometer). In the case of automatic or electronic blood pressure meter, oscillometric methods are usually employed due to its improved signal to noise ratio capabilities and no need of detection of Korotkoff (blood flow) sounds. Furthermore, this method allows visualizing blood pressure wave or pulse wave, and it improves the medical evaluation of a subject.

The cuff size for an upper arm type blood pressure monitor is an important consideration. The ideal cuff should have a bag width at least 40% of the arm circumference, and double of the width is recommended for the length of the bag. For a small adult with an arm circumference of 22 to 26 cm, 12 cm bag width is recommended, while for a standard adult with an arm circumference of 27 to 34 cm (or more), 16 cm bag width is recommended [NPL (Non Patent Literature) 1, page 705]. However, these considerations are probably based on cuffs composed of single air bag (bladder) suffering from cuff-edge problems.

A cuff with a bag having 12 cm width is used in most of the medical checks. These checks are usually fast and less than 5 minutes. Even though, comfortability is not an issue during medical checks, a cuff width around 12 cm is not comfortable for daily uses and/or for continuous blood pressure measurements, i.e. ambulatory blood pressure measurement (ABPM). It is a known fact that blood pressure measurement results can be affected by white-coat hypertension and cause erroneous results and treatments. The blood pressure measurements out of hospitals, at homes or during daily life are recommended for more reliable results especially to predict the risks of cardiovascular events and to diagnose the white-coat hypertension [NPL 1]. However, current cuffs have large width and they are stressful to the user during daily life. A smaller cuff width without sacrificing the accuracy is appreciated for daily life measurements and it remains as a problem.

Mercury type upper arm blood pressure monitor has been accepted as a gold standard [NPL 1]. Typical commercially available cuff of mercury type blood pressure monitor has an inflatable/deflatable air bag (or occlusion component) width around 12 cm, and its cross section on a body portion (e.g. an arm or leg) of human (or animal) is similar to ellipsoid. The proximal side (near to the heart) is called as upstream side and the distal side (near to the hand or foot) is called as downstream side. The occlusion component is pressurized to occlude the artery and the blood pressure is measured based on the oscillations caused by oscillations in the underlying artery.

During the pressurization of the cuff, however, the heart continues to pump the blood and it hits to the walls of the occluded artery under the cuff. The blood flow from the heart side reflects back and causes upstream flows in the proximal side. The cuff under the pressurization resembles an ellipsoid in cross-section, and it loses the efficiency of the contact with skin at the edges. This is known as cuff-edge effect. It causes a non-uniform pressure distribution over the artery leading to a partial occlusion or a narrower occlusion of the artery around the center of the cuff. Due to cuff-edge effects, the effective occlusion width is smaller than that of the cuff along the axis around which the cuff is wrapped.

Although the aforementioned width with its pressure distribution characteristic in typical cuffs is tolerable, decreasing the width enhances cuff-edge effect, and this will probably cause erroneously high readings [NPL 2] due to probably incompletely and/or non-uniformly transmitted pressure to the artery under a narrower cuff or mis-cuffing. Therefore, if the pressure can be completely or uniformly transmitted to or distributed over the artery under the cuff by reducing those cuff-edge effects, smaller cuff width for standard adults is realizable and applicable with enough measurement accuracy.

We previously presented to reduce significantly the foregoing problem and to enable smaller cuff width (for a standard adult) leading to relatively more comfortable, low volume and compact wearable medical devices from ABPM applications to consumer applications. It is achieved such that a blood pressure cuff comprising a relatively low volume (narrower) occlusion component is configured to occlude the artery, a pulse wave detection component is configured to detect pulse wave or blood pressure oscillations, and a compliance material is configured to be placed between said occlusion component and said pulse wave detection component in order to disperse the pressure over the artery to reduce cuff-edge problems (or mis-cuffing) caused by said occlusion component to achieve a low volume or narrower cuff width.

### CITATION LIST

### Non Patent Literature

[NPL 1] Thomas G. Pickering et al., “Recommendations for blood pressure measurement in humans and experimental animals. Part 1: Blood pressure measurement in humans: A statement for professionals from the subcommittee of professional and public education of the American Heart Association council of high blood pressure research”, Circulation, 111, 697-716, 2005
[NPL 2] M. Ramsey, “Blood pressure monitoring: Automated oscillometric devices”, J. Clin. Monit., 7, 56-67, 1991

### SUMMARY OF INVENTION

### Technical Problem

Occlusion component to occlude the artery and pulse wave detection component to detect blood pressure oscillations are different in volume. While continuous pumping improves inflation of occlusion component having relatively bigger size, it causes over-inflation of pulse wave detection component due to excessive delivery of fluid to a smaller volume. This situation in pulse wave detection component causes inaccurate or over-estimated blood pressure readings due to big changes in small volume.

### Solution to Problem

Fluidic connection between occlusion component and pulse wave detection component or to the pulse wave detection component is controlled and adjusted.

### Advantageous Effects of Invention

With its narrower width and so the low volume, the blood pressure cuff according to the present invention achieves medically accurate and precise blood pressure readings similar to its commercial counterparts, i.e. 12 cm width, with great potentials of more comfortable medical devices and ABPM applications.

### BRIEF DESCRIPTION OF DRAWINGS

FIG. 1 Different system connection possibilities to control and adjust pressurization and depressurization;

FIG. 2 A cross-sectional view of the first embodiment of the blood pressure cuff with independent pressurization/depressurization sources along an axis around which the cuff is wrapped;

FIG. 3 A cross-sectional view of the second embodiment of the blood pressure cuff with fluidic/acoustic impedance on the way to pulse wave detection component along an axis around which the cuff is wrapped;

FIG. 4 An experimental blood pressure wave data, its first order differentiation and over-estimated blood pressure readings of second embodiment in FIG. 3;

FIG. 5 Magnification of FIG. 4 between diastolic and systolic blood pressure values;

FIG. 6 A cross-sectional view of the third embodiment of the blood pressure cuff with fluidic/acoustic impedance and a controllable multi-port valve on the way to pulse wave detection component along an axis around which the cuff is wrapped;

FIG. 7 An experimental blood pressure wave data, its first order differentiation, and estimated blood pressure readings within medical approval accuracy of the third embodiment in FIG. 6;

FIG. 8 A statistical data showing blood pressure readings within medical approval accuracy of the third embodiment in FIG. 6;

FIG. 9 A cross-sectional view of the fourth embodiment of the blood pressure cuff where a multi-port controllable valve is used among pumping source, occlusion component and pulse wave detection component;

FIG. 10-A Some examples or combinations of parallel pressurization of both components;

FIG. 10-B Some examples or combinations of serial pressurization of both components;

### DESCRIPTION OF EMBODIMENTS

Exemplary embodiments for carrying out the present invention will be described using drawings in the following. However, although exemplary technical limitations for carrying out the present invention are applied to the exemplary embodiments described below, the scope of the invention is not limited to below.

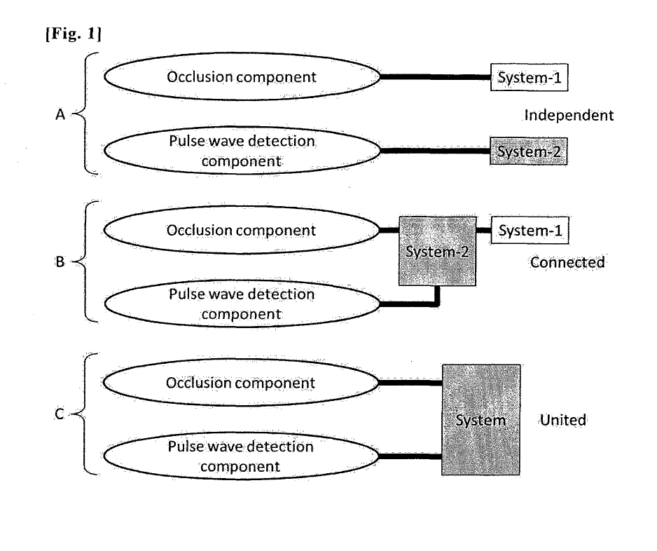

The structure of the blood pressure cuff containing occlusion component to occlude the artery to block the blood flow, which is preferably an inflatable/deflatable bag or bladder, and a pulse wave detection component to detect or sense pulse wave or blood pressure waves or oscillations in the artery, which is preferably an inflatable/deflatable bag or bladder, is shown in FIG. 1. The pulse wave detection component can have another fluidic connection to pressure sensor to measure the pressure level in itself, and it is not shown in the figure for simplicity.

Both occlusion component and pulse wave detection component can be designed such that they can be connected as independent systems (FIG. 1-A), connected systems (FIG. 1-B), and united systems (FIG. 1-C). The systems in the figure can contain fluidic and/or acoustic components, devices and tools such as pumps, valves, handbulbs, screws, tubes, connectors, fluidic/acoustic impedances, and so on. The systems can contain electrical or electronic circuitry too.

FIG. 1-A shows independent systems, where system-1 is connected to occlusion component and system-2 is connected to pulse wave detection component. The reason for this is that occlusion component and pulse wave detection component can be significantly different in volume. In reality, to occlude the artery underlying the blood pressure cuff occupies huge volume and space. Pulse wave detection component is relatively smaller than occlusion component. This volume difference causes an independent volume and pressure control and/or adjustment.

FIG. 1-B shows another possibility. The problem is to control and adjust excessive fluid flowing into pulse wave detection component. In the fluidic way to the pulse wave detection component, system-2 can be positioned such that the system-2 can have fluidic connection to occlusion component and system-1.

In FIG. 1-C, system-1 and system-2 can be united to provide the same duties mentioned in FIGS. 1-A and 1-B.

### First Exemplary Embodiment

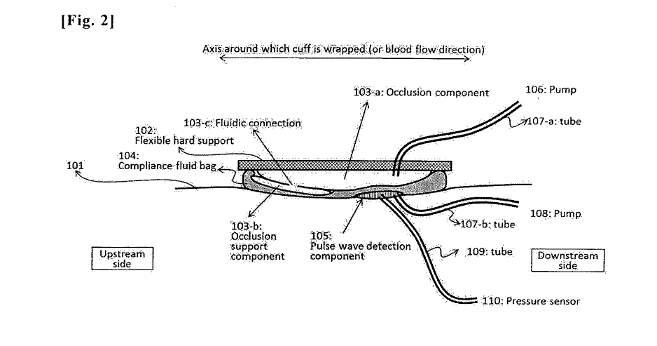

The blood pressure cuff of the first exemplary embodiment with independent pumping sources is shown in FIG. 2. (It is not shown, but pumping sources can contain electrical circuitry and depressurization unit including an escape valve.) It shows cross-section of the cuff wrapped around a body portion 101, preferably an arm. It contains an occlusion component 103-a, preferably an inflation/deflating bag, in order to occlude the underlying artery or to block the blood flow in the artery. Its volume is relatively smaller than its commercial counterparts, and this makes it narrower and less dependent on pump capabilities, which leads to more compact blood pressure meters or ABPMs.

Occlusion component 103-a may have a branch or braches like occlusion support component 103-b, to support the suppression of the upstreams at proximal (heart) side for a better pulse wave detection and improvement of signal to noise ratio. It is connected to occlusion component 103-a via a fluidic connection 103-c. Pulse wave or blood pressure oscillations in the underlying artery is detected by pulse wave detection component 105, which is preferably an inflation/deflation bag. The pulse wave detection component 105 is significantly smaller in size compared to occlusion component 103, and therefore they are pressurized and depressurized by independent sources via 106 and 108.

Between occlusion component 103 and pulse wave detection component 105, compliance fluid bag 104 is placed. It contains fluids, preferably liquids or gels. The compliance fluid bag 104 improves the compliance between occlusion component 103 and the body portion 101, and it enhances uniform pressure distribution on underlying artery. By using the compliance fluid bag 104, it is possible to use smaller volumes for occlusion component 103 which leads to lower space, lower pumping necessities on the pumps, and more compact ABPMs.

To improve the compliance between occlusion component 103 and the body portion 101, the compliance fluid bag 104 is appreciated to have a bigger width and length compared to occlusion component 103.

In order to achieve a better occlusion, it is necessary to limit the freedom of the occlusion component 103 and compliance fluid bag 104 against the body portion 101. This is achieved by a flexible hard support 102 place on occlusion component 103, compliance fluid bag 104 and pulse wave detection component 105.

Referring to FIG. 2, independent pumping sources such as pump 106 (depressurization capability not shown for simplicity) connected to occlusion component 103 via a tube 107-a, and independent pump 108 (depressurization capability now shown for simplicity) connected to pulse wave detection component 105 via tube 107-b. Pumps can be different in speed, size, and mechanism (including manual ones such as a handbulb). The pressure inside pulse wave detection component 105 can be measured by a pressure sensor 110 via tube 109.

Pulse wave detection component 105 can be positioned on the distal side or downstream side of the blood pressure cuff to achieve an improved signal to noise ratio. However, it is preferably positioned under the compliance fluid bag 104.

### Second Exemplary Embodiment

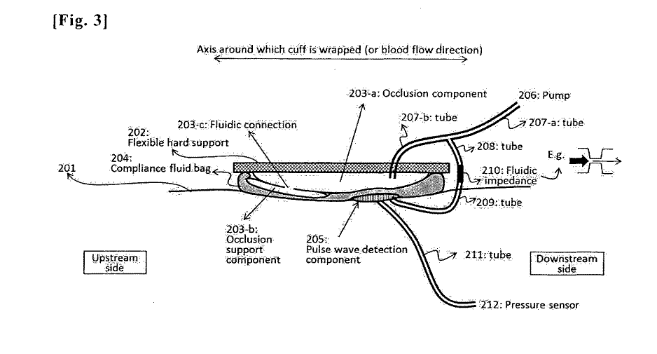

The second exemplary embodiment of the blood pressure cuff is shown in FIG. 3. (It is not shown, but pumping source can contain electrical circuitry and depressurization unit including an escape valve.) It shows cross-section of the cuff wrapped around a body portion 201, preferably an arm. It contains an occlusion component 203-a, preferably an inflation/deflation bag, in order to occlude the underlying artery or to block the blood flow in the artery. Its volume is relatively smaller than its commercial counterparts, and this makes it narrower and less dependent on pump capabilities, which leads to more compact blood pressure meters or ABPMs.

Occlusion component 203-a may have a branch or braches like occlusion support component 203-b, to support the suppression of the upstreams at proximal (heart) side for a better pulse wave detection and improvement of signal to noise ratio. It is connected to occlusion component 203-a via a fluidic connection 203-c. Pulse wave or blood pressure oscillations in the underlying artery is detected by pulse wave detection component 205, which is preferably an inflation/deflation bag.

Between occlusion component 203 and pulse wave detection component 205, compliance fluid bag 204 is placed. It contains fluids, preferably liquids or gels. The compliance fluid bag 204 improves the compliance between occlusion component 203 and the body portion 201, and it enhances uniform pressure distribution on underlying artery. By using the compliance fluid bag 204, it is possible to use smaller volumes for occlusion component 203 which leads to lower space, lower pumping necessities on the pumps, and more compact ABPMs.

To improve the compliance between occlusion component 203 and the body portion 201, the compliance fluid bag 104 is appreciated to have a bigger width and length compared to occlusion component 203.

In order to achieve a better occlusion, it is necessary to limit the freedom of the occlusion component 203 and compliance fluid bag 204 against the body portion 201. This is achieved by a flexible hard support 202 placed on occlusion component 203, compliance fluid bag 204 and pulse wave detection component 205.

Referring to FIG. 3, pumping source pump 206 (depressurization capability not shown for simplicity) is fluidically connected to both occlusion component 203 via tubes 207-a and 207-b and connected to pulse wave detection component 205 via tubes 208 and 209 with a fluidic impedance 210 or resistance. The pulse wave detection component 205 is significantly smaller in size compared to occlusion component 203, and therefore they are separated by fluidic impedance 210 by using the same pumping source pump 206. Since, this resistance being a hollow tube, which is very small in cross section compared to tubes in the FIG. 3, adjusts the amount of the flow. Therefore, excessive fluid flow into pulse wave detection component 205 is prevented to some extent to eliminate over-estimated blood pressure readings.

Pump 206 can be electrical or mechanical such as a manual handbulb. The pressure inside pulse wave detection component 205 can be measured by a pressure sensor 212 via tube 211.

Pulse wave detection component 205 can be positioned on the distal side or downstream side of the blood pressure cuff to achieve an improved signal to noise ratio. However, it is preferably positioned under the compliance fluid bag 204.

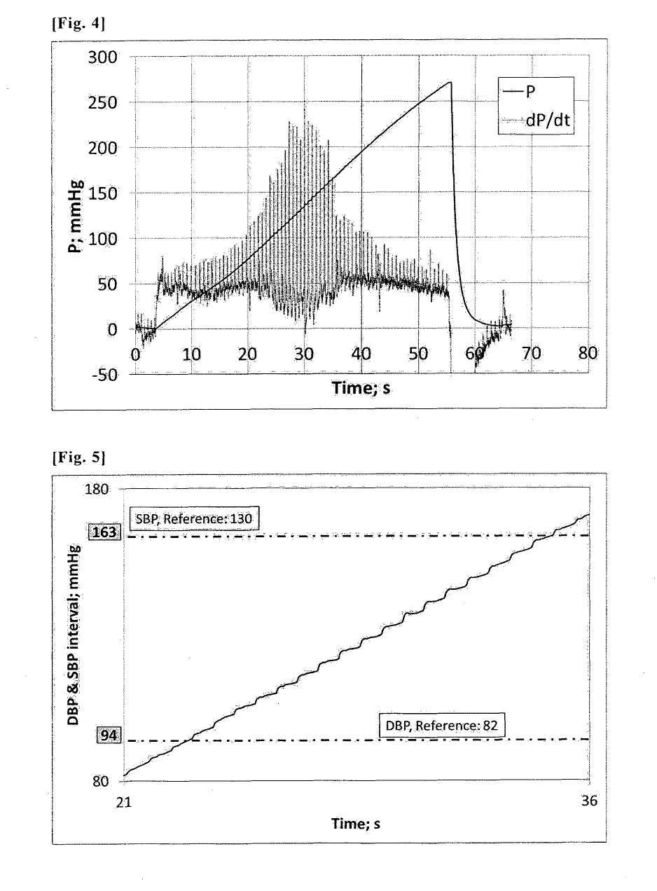

Referring to FIG. 3, a prototype device is constructed using commercial fluidic impedance. FIG. 4 shows experimental results with blood pressure (P) raw-data (solid line) in pulse wave detection component 205 and its first-order differentiation (dP/dt, dotted line). According to this data, the prototype device can detect the pulse wave very clearly. The magnified image is presented in FIG. 5, where the internal between systolic blood pressure (SBP) and diastolic blood pressure (DBP) is magnified. Oscillations are clear. The FIG. 5 includes commercial blood pressure readings as a reference where SBP is 130 mmHg, and DBP is 82 mmHg, which are safe values. However, although fluidic impedance 210 is employed to adjust fluid amount going to pulse wave detection component 205, SBP of the prototype device is estimated as 163 mmHg, and DBP of the prototype device is estimated as 94 mmHg. SBP is over-estimated as high as 33 mmHg, and DBP is over-estimated as high as 12 mmHg.

This over-estimation problem is related to the continuous or excessive pumping of fluid into the pulse wave detection component 205. Due to excessive amount of fluid, and relatively smaller size or volume, small fluctuations can cause big pressure deviations increasing the error. If the amount of flow or the flow speed can be controlled or adjusted, accurate values can be reached. In addition, increasing the value of impedance can be another solution.

To control and adjust the fluid flow into pulse wave detection component, another embodiment is proposed next by employing a valve.

### Third Exemplary Embodiment

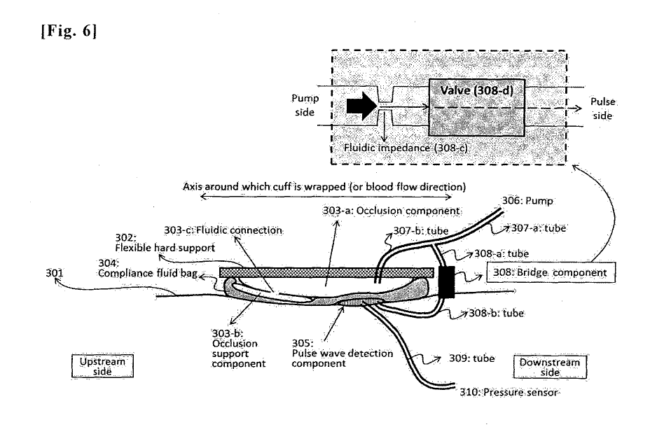

The third exemplary embodiment of the blood pressure cuff is shown in FIG. 6. (It is not shown, but pumping source can contain electrical circuitry and depressurization unit including an escape valve.) It shows cross-section of the cuff wrapped around a body portion 301, preferably an arm. It contains an occlusion component 303-a, preferably an inflation/deflating bag, in order to occlude the underlying artery or to block the blood flow in the artery. Its volume is relatively smaller than its commercial counterparts, and this makes it narrower and less dependent on pump capabilities, which leads to more compact blood pressure meters or ABPMs.

Occlusion component 303-a may have a branch or braches like occlusion support component 303-b, to support the suppression of the upstreams at proximal (heart) side for a better pulse wave detection and improvement of signal to noise ratio. It is connected to occlusion component 303-a via a fluidic connection 303-c. Pulse wave or blood pressure oscillations in the underlying artery is detected by pulse wave detection component 305, which is preferably an inflation/deflation bag.

Between occlusion component 303 and pulse wave detection component 305, compliance fluid bag 304 is placed. It contains fluids, preferably liquids or gels. The compliance fluid bag 304 improves the compliance between occlusion component 303 and the body portion 301, and it enhances uniform pressure distribution on underlying artery. By using the compliance fluid bag 304, it is possible to use smaller volumes for occlusion component 303 which leads to lower space, lower pumping necessities on the pumps, and more compact ABPMs.

To improve the compliance between occlusion component 303 and the body portion 301, the compliance fluid bag 304 is appreciated to have a bigger width and length compared to occlusion component 303.

In order to achieve a better occlusion, it is necessary to limit the freedom of the occlusion component 303 and compliance fluid bag 304 against the body portion 301. This is achieved by a flexible hard support 302 placed on occlusion component 303, compliance fluid bag 304 and pulse wave detection component 305.

Referring to FIG. 6, pumping source pump 306 (depressurization capability not shown for simplicity) is fluidically connected to both occlusion component 303 via tubes 307-a and 307-b and connected to pulse wave detection component 305 via tubes 308-a and 308-b via a component called as bridge component 308 (functioning as a bridge between occlusion component 303 and pulse wave detection component 305) which contains fluidic impedance 308-c and a valve 308-d (preferably electrical). The pulse wave detection component 305 is significantly smaller in size compared to occlusion component 303, and therefore they are separated by bridge component 308 in order to use the same pump 306. This resistance being a hollow tube adjusts the amount of the flow. The control of the flow is done by switching the valve 308-d ON and OFF. Therefore, excessive fluid flow into pulse wave detection component 305 is prevented to eliminate over-estimated blood pressure readings.

Pump 306 can be electrical or mechanical such as a manual handbulb. The pressure inside pulse wave detection component 305 can be measured by a pressure sensor 310 via tube 309.

Pulse wave detection component 305 can be positioned on the distal side or downstream side of the blood pressure cuff to achieve an improved signal to noise ratio. However, it is preferably positioned under the compliance fluid bag 304.

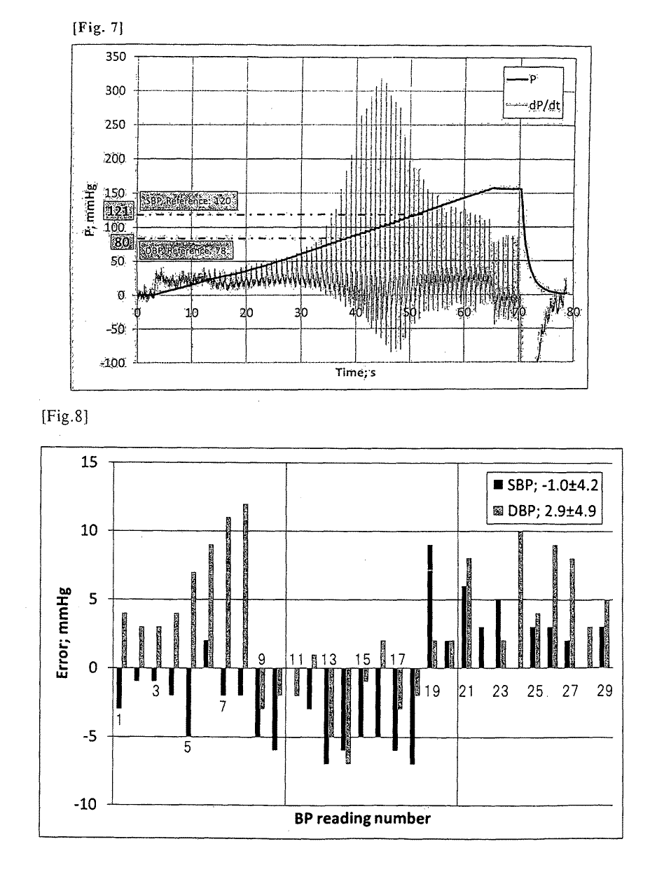

Referring to FIG. 6, a prototype device is constructed using commercial fluidic impedance and electrical solenoid valve. FIG. 7 shows experimental results with blood pressure (P) raw-data (solid line) in pulse wave detection component 305 and its first-order differentiation (dP/dt, dotted line). According to this data, the prototype device can detect the pulse wave, and oscillations are clear. The FIG. 7 includes commercial blood pressure readings as a reference where SBP is 120 mmHg, and DBP is 78 mmHg, which are safe values. The estimated SBP of the prototype device is 121, and estimated DBP of the prototype device is 80 mmHg. Both readings are highly accurate and they are in medical approval accuracy (±5 mmHg).

Here, the point where valve 308-d in bridge component 308 switched OFF has critical importance to control and adjust the fluid amount flowing into the pulse wave detection component 305. In this experiment, it is around 25 mmHg. This switching point (or the threshold value) of the valve to block the fluid is appreciated to be less that the DBP of the subject, i.e. preferably less than 50 mmHg.

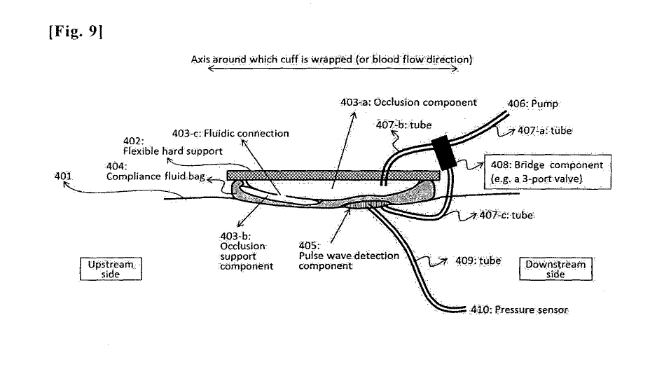

The number of the experiments for blood pressure readings with another subject is increased to 29 (FIG. 8) by using an 8 cm-wide blood pressure cuff (without textile). The occlusion component 303 was around 6 cm in width, the compliance fluid bag 304 was about 7.5 cm, and the flexible hard support 302 was around 8 cm in width which are lower and more compact compared to commercial products. According to the statistical results shown in FIG. 8, errors (or deviations) in SBP readings are −1.0±4.2 mmHg, and errors in DBPs are 2.9±4.9. They are highly accurate and precise where medical approval accuracy is ±5.0±8.0. The results prove that by controlling and adjusting fluid flow into pulse wave detection component, accurate blood pressure estimations are achievable in more compact blood pressure cuffs.

### Fourth Exemplary Embodiment

The fourth exemplary embodiment of the blood pressure cuff is shown in FIG. 9. (It is not shown, but pumping source can contain electrical circuitry and depressurization unit including an escape valve.) It shows cross-section of the cuff wrapped around a body portion 401, preferably an arm. It contains an occlusion component 403-a, preferably an inflation/deflating bag, in order to occlude the underlying artery or to block the blood flow in the artery. Its volume is relatively smaller than its commercial counterparts, and this makes it narrower and less dependent on pump capabilities, which leads to more compact blood pressure meters or ABPMs.

Occlusion component 403-a may have a branch or braches like occlusion support component 403-b, to support the suppression of the upstreams at proximal (heart) side for a better pulse wave detection and improvement of signal to noise ratio. It is connected to occlusion component 403-a via a fluidic connection 403-c. Pulse wave or blood pressure oscillations in the underlying artery is detected by pulse wave detection component 405, which is preferably an inflation/deflation bag.

Between occlusion component 403 and pulse wave detection component 405, compliance fluid bag 404 is placed. It contains fluids, preferably liquids or gels. The compliance fluid bag 404 improves the compliance between occlusion component 403 and the body portion 401, and it enhances uniform pressure distribution on underlying artery. By using the compliance fluid bag 404, it is possible to use smaller volumes for occlusion component 403 which leads to lower space, lower pumping necessities on the pumps, and more compact ABPMs.

To improve the compliance between occlusion component 403 and the body portion 401, the compliance fluid bag 404 is appreciated to have a bigger width and length compared to occlusion component 403.

In order to achieve a better occlusion, it is necessary to limit the freedom of the occlusion component 403 and compliance fluid bag 404 against the body portion 401. This is achieved by a flexible hard support 402 placed on occlusion component 403, compliance fluid bag 404 and pulse wave detection component 405.

Referring to FIG. 9, pumping source pump 406 (depressurization capability not shown for simplicity) is fluidically connected to both occlusion component 403 via tubes 407-a and 407-b and to pulse wave detection component 405 via tube 407-c and through a component called as bridge component 408 which can contain a multi-port electrical valve (e.g. a 3-port valve). The pulse wave detection component 405 is significantly smaller in size compared to occlusion component 403, and therefore they are separated by bridge component 408 in order to use the same pump 406. The control of the flow is done by switching the bridge component 408 ON and OFF. Therefore, excessive fluid flow into pulse wave detection component 405 can be controlled.

Pump 406 can be electrical or mechanical such as a manual handbulb. The pressure inside pulse wave detection component 405 can be measured by a pressure sensor 410 via tube 409.

Pulse wave detection component 405 can be positioned on the distal side or downstream side of the blood pressure cuff to achieve an improved signal to noise ratio. However, it is preferably positioned under the compliance fluid bag 404.

Usual 3-port valve opens one port in a given of time, and closes the other port at that time. Therefore, fluid pumping into both of the components (403 and 405) is not possible. Therefore, instead of parallel pumping or pumping fluid into both components at the same time, serial or alternating pumping can be considered.

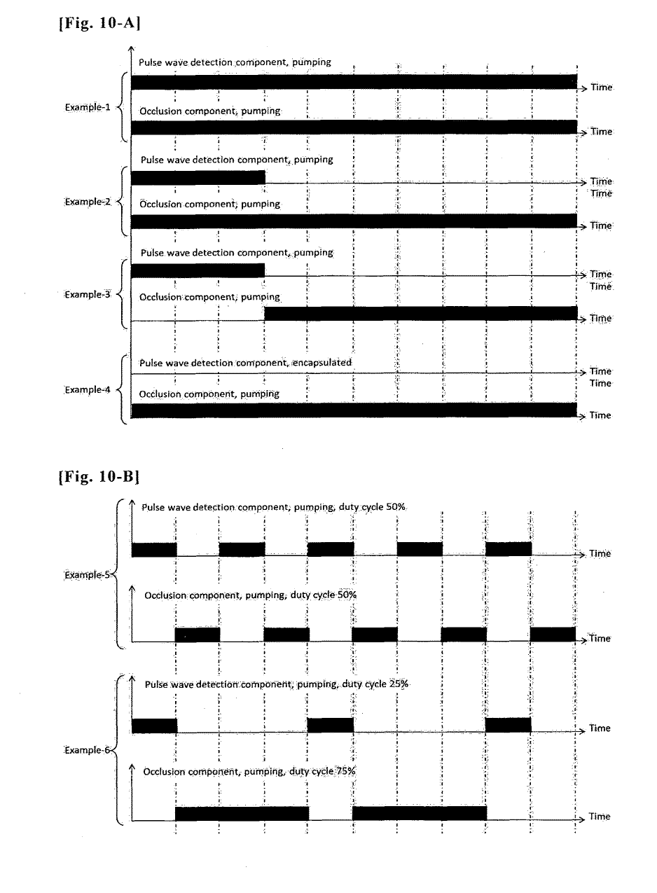

The last 2 figures, 10-A and 10-B, show some pumping examples and control protocols of the pumping. FIG. 10-A shows parallel pumping examples, where both occlusion component and pulse wave detection component is pumped at the same time.

Example-1 (FIG. 10-A) shows a parallel pumping example. Both components are pumped, without switching ON/OFF the fluid into the pulse wave detection component until a complete occlusion of the artery is reached. This example actually is used in an experiment whose results are shown in FIGS. 4 and 5.

Example-2 (FIG. 10-A) shows another parallel pumping example. It is such that both components are pumped first until a threshold value (a specific value) where pulse wave detection component is switched OFF or blocked. But, after that value occlusion component continues to be pumped. This example corresponds to the experiment whose result is shown in FIG. 7.

Example-3 (FIG. 10-A) shows another parallel pumping example. It is such that pulse wave detection component is pumped first until a threshold value (a specific value) and then it is switched OFF or blocked. Just after switching occlusion component starts to be pressurized by pumping from a zero or an initial value.

Example-4 (FIG. 10-A) shows another parallel pumping example. It is such that pulse wave detection component is sealed or encapsulated by a given amount of fluid (air or liquid such as silicone oil) and volume, and it is connected to pressure sensor. Occlusion component is pumped only.

Example-5 (FIG. 10-B) shows a serial or alternating pumping example, i.e. one component in a given of time. Both components are pumped with the same duty cycle. To switch from one component to the other, electrical and fluidic circuitry can be used. Example-6 shows that duty cycle (or pumping time of a component) can be different due to significant volume differences of the components.

Apart from those, it is possible to apply or to find different control protocols, and/or combination of the control protocols mentioned above. For example, a prototype shown in FIG. 3 can be employed by a control protocol shown in example-6 in FIG. 10-B. Or, a prototype shown in FIG. 9 can be employed by a control protocol shown in example-6 in FIG. 10-B. Those examples can be increased without departing from the basic idea, which is pulse wave sensing bag is smaller in volume compared to occlusion component, and control or adjustment of the fluid flow into or fluid amount in the pulse sensing bag has critical importance for an accurate blood pressure reading.

### INDUSTRIAL APPLICABILITY

This invention can be applied to the blood pressure meters and ABPMs.

### REFERENCE SIGNS LIST

101, 201, 301, 401, subject (body portion of arm or leg)
102, 202, 302, 402, flexible hard support
103-a, 203-a, 303-a, 403-a, occlusion component
103-b, 203-b, 303-b, 403-b, occlusion support component
103-c, 203-c, 303-c, 403-c, fluidic connection
104, 204, 304, 404, compliance fluid bag
105, 205, 305, 405, pulse wave detection component
106, 108, 206, 306, 406, pump
107, 109, 207, 208, 209, 211, 307, 308, 309, 407, 409, tube
110, 212, 310, 410, pressure sensor
210, 308-c, fluidic impedance
308-d, valve
308, 408, bridge component

1. A blood pressure cuff comprising,
an occlusion component configured to occlude the artery,
and
a pulse wave detection component configured to detect blood pressure,
where, said occlusion component and said pulse wave detection component are configured to be controlled by independent systems, which can contain electrical/electronic circuitry, energy unit, fluidic and/or acoustic components, devices or tools to achieve pressurization and depressurization.

2. The blood pressure cuff of claim 1,
wherein, said systems can be fluidically connected such that one of said systems can adjust the fluid amount flowing into said pulse wave detection component.

3. The blood pressure cuff of claim 1,
wherein said systems are united, where one port of said system control fluid flow into said occlusion component and another port of said system control fluid flow into said pulse wave detection component.

4. The blood pressure cuff of claim 2,
wherein said system connected to said pulse wave detection component can contain multiple inlets/outlets with a fluidic impedance with/without a controllable valve to control the amount of the air flow and its blocking.

5. The blood pressure cuff of claim 2,
wherein said system connected to said pulse wave detection component can contain multi-port (2 or more ports) controllable valve.

6. The blood pressure cuff of claim 1, wherein said systems can be controlled such that said occlusion component and said pulse wave detection component can be pressurized in a parallel or in a serial manner with different duty cycle combinations.

7. The blood pressure cuff of claim 1, wherein said systems can be controlled such that said pulse wave detection component can be blocked during pressurization.

8. The blood pressure cuff of claim 1, wherein said systems can be controlled such that said pulse wave detection component can be pressurized first and then blocked, and occlusion component can be pressurized next.

9. The blood pressure cuff of claim 1, wherein said systems can be controlled such that said pulse wave detection component can be encapsulated by a fluid, and occlusion component can be pressurized only.

10. A blood pressure meter, wherein a blood pressure cuff in claim 1 is included.
